(19) United States
(12) Patent Application Publication
(10) Pub. No.: US 2004/0186117 A1
(43) Pub. Date: Sep. 23, 2004

(54) Marker

(76) Inventors: Paul Workman, Belmont (GB); Steven R. Whittaker, Belmont (GB)

Correspondence Address: LAHIVE & COCKFIELD, LLP., 28 STATE STREET, BOSTON, MA, 02109, US

(21) Appl. No.: 10/632,567

(22) Filed: Jul. 31, 2003

Related U.S. Application Data
(63) Continuation of application No. PCT/GB02/00427, filed on Jan. 31, 2002.

(30) Foreign Application Priority Data
Jan. 31, 2001 (GB) 0102480.1

(51) Int. Cl. A61K 31/522

(52) U.S. Cl. 514/263.37

(57) ABSTRACT

The present invention relates to a pharmacodynamic marker for the candidate 2,6,9-tri-substituted purine known as roscovitine. The identity of this marker facilitates the convenient identification of roscovitine activity both in vitro and in vivo.

### RELATED APPLICATIONS

This application is a continuation of International Application No. PCT/GB02/00427, filed Jan. 31, 2002, which claims priority to a United Kingdom Patent Application No. 0102480.1 filed Jan. 31, 2001, the entire contents of each of which are incorporated herein by reference.

### BACKGROUND

The present invention relates to a pharmacodynamic marker for the candidate 2,6,9-tri-substituted purine known as roscovitine. The identity of this marker facilitates the convenient identification of roscovitine activity both in vitro and in vivo.

The 2,6,9-tri-substituted purines are becoming a well studied class of compound showing promise as cyclin dependent kinase inhibitors (CDKI's) of use in the treatment of proliferative disorders such as cancers and leukemias. Fischer P & Lane D (Curr Med Chem (2000) vol 7 page 1213) provides a detailed review of CDKI's, their origins and described activities. The compound (R)-2-[(1-ethyl-2-hydroxyethyl)amino]-6-benzylamino-9-isopropylpurine, known as R-roscovitine was first described in WO97/20842 (Meijer L et al) and has since been developed as a promising candidate anti-cancer agent.

In the development of such agents, extensive pharmacokinetic and pharmacodynamic investigations must be undertaken inorder to understand the actual mechanism of action upon administration and satisfy the regulatory authorities requirements as to toxicity and dosing. Such analysis is based upon the complex biochemistry of the cell cycle control system and detailed studies undertaken in the pre-clinical phase of drug development to ascertain the particular mode of activity of the candidate drug.

Of particular advantage in the pharmacokinetic and pharmacodynamic investigations is the identity of a specific marker of activity for the candidate drug. This is particularly the case in circumstances where several related compounds may be administered simultaneously for pharmacokinetic/pharmacodynamic evaluation. Such a protocol permits for example, a single animal model to be used to test three or more compounds, the investigator then being able to monitor the breakdown products and relate them to one of the individual compounds administered. Such protocols, described as cassette dosing are particularly valuable in the present environment with public concern as to the number of animals used in medical research (Raynaud FI et al. Abstract 5179, AACR, San Francisco 2000, and AACR, New Orleans 2001).

### BRIEF DESCRIPTION OF THE DRAWINGS

FIG. 1. Inhibition of RB phosphorylation and induction of ERK phosphorylation by R-roscovitine. Asynchronous HT29 and KM12 cells were exposed to increasing concentrations of R-roscovitine for 24 h. Cell lysates were prepared and 25 μg of protein was resolved by SDS-PAGE and subjected to Western Blotting for total RB protein, phosphorylated-RB at Ser780, total ERK2 and phospho-ERK1/2.

FIG. 2. Time course of inhibition of RB phosphorylation and induction of ERK phosphorylation by R-roscovitine. Asynchronous HT29 and KM12 cells were exposed to 50 μM R-roscovitine for the indicated times. Cell lysates were prepared and 25 μg of protein was resolved by SDS-PAGE and subjected to Western Blotting for total RB protein, phosphorylated RB and Ser608, Ser780 and Ser807/811, total ERK2 and phospho-ERK1/2.

FIG. 3. Effect of different CDK inhibitor chemotypes upon RB and ERK phosphorylation. Asynchronous HT29 cells were treated with iso-effective concentrations of either olomoucine (174 μM), R-roscovitine (48 μM), purvalanol A (12 μM), flavopiridol (480 nM) or alsterpaullone (1350 nM) for 24 h. Cell lysates were prepared and 25 μg of protein was resolved by SDS-PAGE and subjected to Western Blotting for total RB protein, phosphorylated-RB at Ser780, total ERK2 and phospho-ERK1/2.

FIG. 4. ERK phosphorylation induced by R-roscovitine is functionally significant. (A) Asynchronous HT29 s were treated with increasing concentrations of R-roscovitine for 24 h. Cell lysates were prepared and 25 or, 50 μg of protein was resolved by SDS-PAGE and subjected to Western Blotting for total ERK2 protein, phospho-ERK1/2 and c-FOS protein. (B) Asynchronous HT29 s were treated with 50 μM R-roscovitine for the indicated times. Cell lysates were prepared and 25 or 50 μg of protein was resolved by SDS-PAGE and subjected to Western Blotting for total ERK2 protein, phospho-ERK1/2 and c-FOS protein.

FIG. 5. Activation of ERK by R-roscovitine is MEK-dependent and functionally significant. (A) Chemical structure and enzyme inhibition profile of U0126, a specific inhibitor of MEK1 and 2. (B) Asynchronous HT29 cells were treated with 50 μM R-roscovitine, 10 μM U0126 or a combination of both for the times indicated. Cell lysates were prepared and 25 or 50 μg of protein was resolved by SDS-PAGE and subjected to Western Blotting for the stated proteins.

FIG. 6. (A) Validation of RB antibodies and blocking reagents. 1: HT29, 2: KM12. 50 μg of protein was loaded per lane and resolved by 6% SDS-PAGE, blotted and probed as described in materials and methods with stated antibodies. (B) Evaluation of lysis procedure shown with response of HT29 cells to 0.1% DMSO, 5 μM and 20 μM purvalanol A treatment for 24 h. Equal volumes of lysate were loaded and resolved by 6% SDS-PAGE, blotted and probed with stated antibodies. Amount of RB signal was compared between total lysate, supermatant and that remaining in the pellet.

FIG. 7. Growth delay analysis of HT29 and KM12 colon tumour lines subjected to increasing doses of roscovitine for 96 h, as determined by SRB assay. IC₅₀ values of 12 μM for KM12 s and 16 μM for HT29 s were determined graphically. Data plotted is the mean of 4 measurements±standard error.

FIG. 8. Western Blot analysis of RB status in asynchronous HT29 s treated with increasing doses of roscovitine for 24 h. 25 μg of protein was resolved by 6% SDS-PAGE, blotted and; probed as described in materials and methods with the stated antibodies. Control lysates were untreated, DMSO lysates were exposed to 0.1% v/v DMSO for 24 h as a vehicle control. Data shown is representative of 3 independent experiments.

FIG. 9. Western Blot analysis of RB status in asynchronous HT29 s treated with 50 μM roscovitine for up to 24 h. 25 μg of protein was resolved by 6% SDS-PAGE, blotted and probed as described in materials and methods with the stated antibodies. Control lysates were untreated, DMSO lysates were exposed to 0.1% v/v DMSO as a vehicle control. Data shown is representative of 2 independent experiments.

FIG. 10. Western Blot analysis of RB status in ascynchronous KM12 s treated with increasing doses of roscovitine for 24 h. 25 μg of protein was resolved by 6% SDS-PAGE, blotted and probed as described in materials and methods with the stated antibodies. Control lysates were untreated, DMSO lysates were exposed to 0.1% v/v DMSO for 24 h as a vehicle control. Data shown is representative of 2 independent experiments.

FIG. 11. (A) Lovo xenografts were either untreated or exposed to 5Gy irradiation, tumours were excised and homogenised in cold lysis buffer. 50 μg of protein was subjected to Western Blotting analysis and probed for total and phospho-RB Ser780. (B) Single and pooled mouse lymphocytes and human lymphocytes were isolated by centrifugation through Lymphoprep™ (Nycomed) and analysed by Western Blotting for total and phospho-RB Ser780. 25 μg of untreated KM12 lysates provide a positive control.

### DETAILED DESCRIPTION OF THE INVENTION

The present invention relates to the observation that erk2 acts as a specific pharmacodynamic (PD) marker for roscovitine, in contrast to related potent CDKI's.

Thus, in a first aspect the invention relates to a method of monitoring the activity of roscovitine comprising

(i) administering roscovitine to a cell, group of cells, an animal model or human, and

(ii) detecting the presence of phosphorylated erk1/2.

Surprisingly, this observation has been found to be unique to roscovitine in that related potent CDKI's such as purvalanol A, though like R-roscovitine in inhibiting the phosphorylation of retinoblastoma (RB) protein and inhibiting cdk2, it fails to induce the phosphorylation of erk1/2. This has also been shown for other agents such as alsterpaullone and flavopiridol.

Erk1/2 is a member of microtubule-associated proteins (the MAP2 kinases) first identified by Boulton et al. (U.S. Pat. Nos. 5,595,904 and 5,776,751) which further describes corresponding antibodies. As used herein and unless specifically expressed to the contrary, the term erk1/2 is used to refer to both erk1 and erk2 together or as is referred to in the Figures as total erk. The same is true when any of these expressions are preceded by the term. “phosphorylated” or “phospho-”. The term erk1/2 is therefore used to refer to these proteins as described in the above-referenced US Patents or, as they may exist in any naturally or mutated isoforms thereof. Thus, if a single patient of sub-group of patients are observed to express an erk1/2-like protein that is phenotypically identical to those described in U.S. Pat. Nos. 5,595,904 and 5,776,751, monitoring of said proteins in their phosphorylated state is not excluded from the present invention.

As used herein the terms and “R-roscovitine” is used to refer to the compound 2-(R)-(1-ethyl-2-hydroxyethylamino)-6-benzylamino-9-isopropylpurine. In it unqualified form the term “roscovitine” is used to include the R-roscovitine, the S enantiomer and racemic mixtures thereof. This compound and its preparation are described in U.S. Pat. No. 6,316,456.

In a second aspect of the invention the phosphorylation state of the RB protein may be used in conjunction with the first aspect of the invention to monitor roscovitine activity. In such an aspect of the invention it is preferred that phosphorylation at the specific serine residues at positions 608 and/or 780 of RB be monitored. Typically, roscovitine will prevent such phosphorylation from occurring.

In a preferred embodiment of the invention roscovitine is administered to a mammal or a human, more preferably a human. When performed on an animal model, the invention is preferably performed on a LoVo or KM12 xenograft mouse model.

In these preferred embodiments, the presence of phosphorylated erk1/2 is preferably detected in tumor cells, lymphocytes, preferably peripheral lymphocytes or buccal mucosal cells.

When the invention is performed ex vivo, it is preferably performed on a group of cells preferably a cell culture. Preferred cell types are selected from HT29, KM12 and HCT116 cells. Alternatively, the cells may be in the form of a histological sample of a tumor biopsy. As such the invention further relates to a method of detecting a proliferative cell in a sample comprising a method as described above.

The methods of the present invention where the levels of phospho-erk1/2 are monitored will preferably involve monitoring the levels prior to administration of roscovitine and the again preferably 2 and/or 4 hours after administration. In a preferred embodiment, the level is monitored again at least 24 hours after administration of roscovitine.

In the preferred embodiments, the level of phosphorylated erk1/2 detected after administration of roscovitine is preferably greater than that detected prior to administration of roscovitine.

The second aspect of the invention relates to the independent monitoring of roscovitine activity by monitoring the levels of phosphorylated RB protein. In a preferred embodiment, this monitoring is conducted together with the monitoring of phosphorylated erk1/2. The level of phosphorylated RB detected after administration of roscovitine is preferably lower than that detected prior to administration of roscovitne.

In a preferred embodiment, the level of phosphorylated erk1/2 is monitored after 2 and/or 4 hours and the level of phosphorylated retinoblastoma (RB) protein is monitored at least 72 hours after administration of roscovitine.

The methods of the present invention may be further utilised in;

(a) methods of assessing suitable dose levels of roscovitine comprising monitoring the degree and rate of erk1/2 phosphorylation after administration of roscovitine to a cell, group of cells, animal model or human,

(b) methods of monitoring the activity of roscovitine in a cassette dosing assay whereby a cocktail of roscovitine and other related CDKI's are administered together and roscovitine activity,

(c) methods of identifying a candidate drug having roscovitine-like activity comprising administering said candidate drug to cell, group of cells, animal model or human and monitoring the presence or absence of erk1/2 phosphorylation or

(c) methods of identifying proliferative cells within a sample exposed to roscovitine comprising monitoring the presence of phospho-erk- 1/2.

Methods such as described in (a) may further comprise correlating the degree and rate of erk1/2 phosphorylation with the known rate of inhibition of either CDK2 or RB phosphorylation by roscovitine at the same dosage, over the same time period.

In a further aspect, the invention relates to the use of phospho-erk 1 and/or phospho-erk 2 in the monitoring of activity of roscovitine utilising any of the methods described above.

In an even further aspect, the invention relates to kits for assessing the activity of roscovitine comprising antibodies for at least one of phospho-erk 1 and/or phospho-erk 2 and optionally antibodies for RB (whole), RB Ser780 or RB Ser608.

Such kits preferably comprise the antibodies for of phospho-erk 1 and/or phospho-erk 2 alone or in combination with one of the RB antibodies preferably the RB Ser608 antibody.

Detection of erk1/2 and phosphorylated (phospho-)erk1/2 and/or RB or phospho-RB may be performed by methods known in the art, particularly by Western blotting. Suitable cell lines for the pharmacodynamic investigation of roscovitine and related compounds include the HT29, KM12, HCT116 cell, and suitable animal models include LoVo or KM12 xenograft mouse models lines (cell lines & models available from S Whittaker, Institute of Cancer Research, Sutton, UK). Antibodies for erk2 are described in the patents of Boulton discussed above and for erk1 are described in U.S. Pat. No. 6,001,580 (Tani). Antibodies for RB are known in the art and are available from S Whittaker supra, antibodies for RB, Ser608 are available from S Mittnacht, Institute of Cancer Research, Sutton, UK).

Typically in cell line investigations a CDK2 inhibitory (IC₅₀) dosage of roscovitine is administered and samples extracted over a 24 or 48 hour time period for example at 2, 4, 12, 24 and 48 hours after administration. Protein samples are isolated, loaded and resolved on SDS-PAGE, blotted and probed for the appropriate marker. When conducting investigation in animal models or humans, a suitable proliferating tissue must be identified as being a source of cells that can be extracted from the animal or human for assessment of roscovitine activity. Suitable tissue includes any proliferating tissue. In particular including a tumor biopsy, but it has now been observed that circulating lymphocytes and cells of the buccal mucosa may also be used. Once extracted, these cells can be treated in a manner identical to that described for cell lines. In most cases a pool of markers including RB, phospho-RB, erk1/2 and phospho-erk1/2 will be employed, but due to the specificity of erk1/2, this will preferably be the sole marker. In some circumstances the RB marker may be used alone.

This embodiment of the invention may be further developed to use the effect of roscovitine on erk1/2 as a tool in dose titration i.e. by monitoring the degree and rate of erk1/2 phosphorylation a suitable dose of roscovitine may be determined. Such analysis may further involve correlation of the degree and rate of erk1/2phosphorylation with the known rate of inhibition of either CDK2 or RB phosphorylation by roscovitine at the same dosage. In this manner, a single measurement of the rate and degree of erk1/2 phosphorylation may be taken as indicative of further activities of roscovitine.

In an even further embodiment of the invention the phosphorylation of erk1/2by a candidate drug may be taken as an indication of its mode of activity in that it may be classified as roscovitine-like.

In accordance with either the first or second aspects, the present invention further relates to a kit for assessing the activity of roscovitine comprising antibodies for at least one of erk1, erk2, RB (whole), RB Ser780 or RB Ser608. Preferably, the kit comprises antibodies for erk1 or erk2 alone or in combination with one of the RB antibodies, preferably RB Ser608. The kits may be used in accordance with any of the hereinbefore described methods for monitoring roscovitine activity, assessing roscovitine dosage or the roscovitine-like activity of a candidate drug.

The observed effect of roscovitine on the levels of phospho-erk1/2 has been shown to result in increased levels of c-FOS and surprisingly decreased levels of cyclin D1. The latter observation is particularly surprising as the expected effect of activating erk phosphorylation would be for cyclin D1 levels to increase. Further aspects of this invention therefore relate to the monitoring of roscovitine activity using methods hereinbefore described with respect to erk1/2 but by monitoring either increased cFOS levels or decreased cyclin D1 levels as compared to their respective levels prior to the administration of roscovitine.

### EXAMPLES

### Section 1

### Materials & Methods

Cell Culture

HT29 (American Type Culture Collection, Manassas, USA) and KM12 (National Cancer Institute, Bethesda, USA) human colon carcinoma cell lines were grown in Dulbecco's Modification of Eagle's Medium (Invitrogen, Paisley, UK) supplemented with 10% FBS (Invitrogen, Paisley, UK) in an atmosphere of 5% CO₂. For drug treatment, cells were counted on a Coulter Z2 (Beckman Coulter, High Wycombe, UK) and 3×10⁶ cells were seeded into a T175 flask and left to attach for 36 h. Compounds were dissolved in DMSO as a 1000×stock and diluted directly into the culture media when required. The total concentration of DMSO in the media did not exceed 0.35% (v/v) during treatments. The R-isomer of roscovitine, olomoucine and purvalanol A were supplied by Cyclacel Ltd., Dundee, UK. Flavopiridol and alsterpaullone were kindly supplied by Dr. Ed Sausville, National Cancer Institute, Bethesda, USA. U0126 was purchased from Promega, Southampton, UK.

Western Blotting

To harvest cells, the medium was removed and cells were incubated with 5 ml trypsin for 5 min at 37° C. to detach them from the plastic. The cells were then pelleted, washed in ice cold PBS and resuspended in ice cold lysis buffer (50 mM HEPES pH7.4, 250 mM NaCl, 0.1% NP40, 1 mM DTT, 1 mM EDTA, 1 mM NaF, 10 mM β-glycerophosphate, 0.1 mM Sodium orthovanadate and 1 Boehringer proteinase inhibitor cocktail tablet per 10 ml of lysis buffer) for 30 mins on ice. Lysates were centrifuged at approx. 18, 000×g for 10 minutes at 4° C. to remove cellular debris. The supernatant was stored at −80° C. prior to use. Protein concentration of lysates was determined using the BCA protein assay (Pierce, Rockford, USA). Proteins were separated by SDS-PAGE using Novex precast Tris-Glycine gels (Invitrogen, Groningen, The Netherlands) and transferred to Immobilon-P membranes (Millipore, Bedford, USA). Membranes were blocked for 1 hour in TBSTM (50 mM Tris pH7.5, 150 mM NaCl, 0.1% Tween 20 (Sigma, Dorset, UK) and 3% milk. Immunoblotting with primary antibodies diluted in TBSTM was performed at 4° C. overnight, followed by a 1 hour incubation with HRP-conjugated secondary antibodies at room temperature. Membranes were washed with ECL reagents and exposed to Hyperfilm (Amersham Pharmacia Biotech, Buckinghamshire, UK). Antibodies used were: C-terminal control total RB 1:5000, phospho-RB Ser780 1:5000, phospho-ERK1/2 1:1000 (Cell Signalling Technologies, Beverly, USA), total RB SC-50 1:2000, total ELK-1 SC-355 1:1000, phospho-ELK-1 SC-8406 1:1000 (Santa Cruz Biotechnology, Santa Cruz, USA), total ERK2 1:10000 (Prof. Chris Marshall, Institute of Cancer Research, London, UK), phospho-RB Ser608 1:2000 (Dr. Sibylle Mittnacht, Institute of Cancer Research, London, UK), phospho-RB Ser807/811 (Sigma, Dorset, UK), goat anti-rabbit and goat anti-mouse HRP-conjugated secondary antibodies 1:5000 (BioRad, Hercules, USA).

Cell Cycle Analysis

3×10⁶ HT29 s were seeded into a 175 cm³ flask and left overnight to attach to the plastic. Drug treatments were performed as above and 30 minutes prior to harvesting, the cells were pulsed with 10 μM BrdU (Sigma, Dorset, UK). Cells were harvested in 5 ml trypsin-versene by incubating at 37° C. for 5 minutes to form a cell suspension, gently pelleted and resuspended in 1 ml ice cold PBS, then fixed by slow addition of 5 ml ice cold 70% ethanol whilst vortexing. For bromodeoxyuridine (BrdU) staining of proliferating cells, cells were resuspended in 2.5 ml 2M HCl with 0.2 mg/ml pepsin at room temperature for 20 minutes to isolate the nuclei. Nuclei were then washed twice with PBS,prior to being resuspended in 0.5 ml PBS, 0.5% Tween 20, 1% BSA containing 20 μl rat anti-BrdU antibody (Immunologicals Direct, Cambridge, UK) for 1 hour at room temperature. Nuclei were then washed with PBS, prior to resuspension in 0.5 ml PBS, 0.5% Tween 20, 1% BSA and 20 μl goat-anti rat IgG whole molecule FITC conjugate (Sigma, Dorset, UK) for 30 minutes at room temperature. Nuclei were then washed with PBS and resuspended in 1 ml 0.02 mg/ml propidium iodide/0.25 mg/ml RNase A and incubated at 37° C. for 30 minutes. Samples were analysed on a Beckman Coulter Elite ESP (Beckman Coulter, High Wycombe, UK) and bivariate cell cycle analysis was performed with WinMidi2.8 software (Scripps Research Institute, La Jolla, USA).

Tumour Xenograft Treatment

Nude mice bearing the KM12 colon tumour cell line as a subcutaneous xenograft in the flank were administered 200 mgkg⁻¹ R-roscovitine orally, 3 times daily for 5 days. R-roscovitine was dissolved in 10% DMSO, 5% Tween 20, 85% 50 mM HCl/saline. For pharmacodynamic profiling, 3 mice were sacrificed per time point. Time points comprised pre-treatment, 3 days vehicle control and 3 days treated (30 min after the last dose), 5 days treated (30 min and 8 h after the last dose) and 24, 48, 72 and 96 h after the last dose. Plasma, tumour, liver and peripheral lymphocytes were recovered from each animal and either homogenised in lysis buffer or snap-frozen in liquid nitrogen and stored at −80° C. For anti-tumour studies, 6 mice were treated with vehicle control alone and 6 mice were treated with R-roscovitine for 5 days as described earlier. Tumour volume was determined from measurement of two orthodiagonal diameters and used to determine anti-tumour activity. For extraction of lymphocytes whole blood was diluted 2:1 in PBS, which was carefully layered onto 1 ml of Lymphoprep™ (Nycomed, Oslo, Norway) in a 15 ml centrifuge tube. Samples were centrifuged at 1000×g for 30 minutes at 4° C. Lymphocytes were removed from the interface between the plasma and the Lymphoprep™, pelleted by centrifugation at 4000×g for 5 minutes at 4° C. and then lysed as above.

Results

R-Roscovitine Inhibits RB Phosphorylation

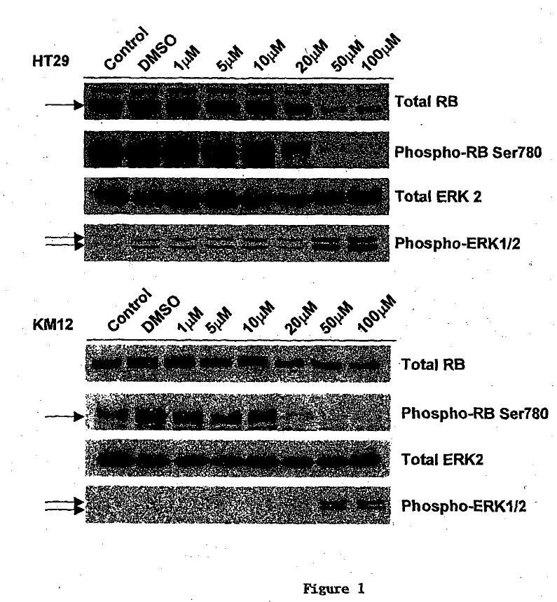

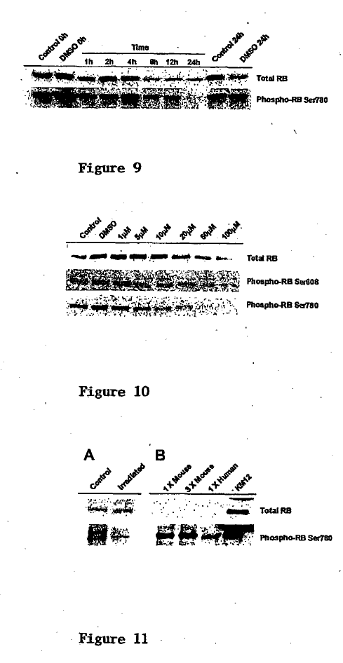

In order to determine whether R-roscovitine was acting as a CDK inhibitor in intact tumour cells, the phosphorylation status of RB protein was determined by Western blotting, using a phospho-specific antibody to Ser780 of RB. This antibody was selected as a representative marker for all tested phosphorylation sites of RB (see Section 2). R-roscovitine treatment for 24 h in asynchronous HT29 and KM12 colon tumour cells resulted in a concentration-dependent loss of RB phosphorylation, with concentrations of 20 μM and greater causing a reduction in phosphorylation at Ser780(FIG. 1). These concentrations correlate well with the IC₅₀ values for these cell lines, 16 μM for HT29 cells and 12 μM for KM12 cells (see Section 2). A general loss of RB phosphorylation is indicated by a switch from a broad band to a thin, more rapidly migrating band for total RB (see Section 2, FIG. 9). Therefore, it can be concluded that R-roscovitine treatment resulted in a loss of RB phosphorylation.

R-Roscovitine Activates ERK1/2

It has been reported that compounds from the tri-substituted purine class of CDK inhibitors may bind to ERK2 (Knockaert, M. et al. (1999). Chemistry and Biology. 7: 411-422). The R-isomer of roscovitine inhibits ERK2 with an IC₅₀ of approximately 1.47 μM. Western blotting of cells exposed to increasing concentrations of R-roscovitine surprisingly demonstrated an increase of phosphorylated ERK1/2 in both HT29 and KM12 cells after 24 h of exposure, indicating likely activation of this kinase (FIG. 1).

Activation of ERK by R-Roscovitine Occurs Prior to Loss of RB Phosphorylation

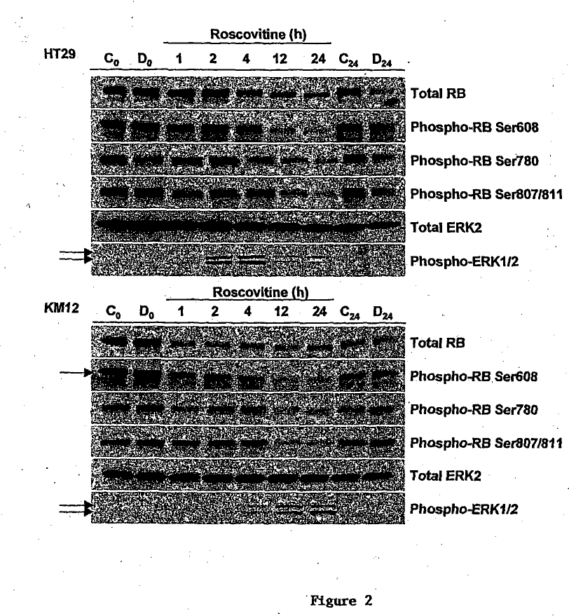

To temporally dissect the events of loss of RB phosphorylation and induction of ERK phosphorylation by R-roscovitine, a time course of 50 μM R-roscovitine was performed upon asynchronous HT29 and KM12 cells (FIG. 2). Dramatic loss of RB phosphorylation occurred from 12-24 h after exposure to the compound. In contrast, induction of ERK phosphorylation was seen as early as 1 h in HT29 cells and peaked at approximately 4 h. KM12 cells showed slower kinetics in the induction of ERK phosphorylation. However, in both cell lines it was observed that activation of ERK preceded loss of RB phosphorylation.

Induction of ERK Phosphorylation is not a Direct Consequence of CDK Inhibition

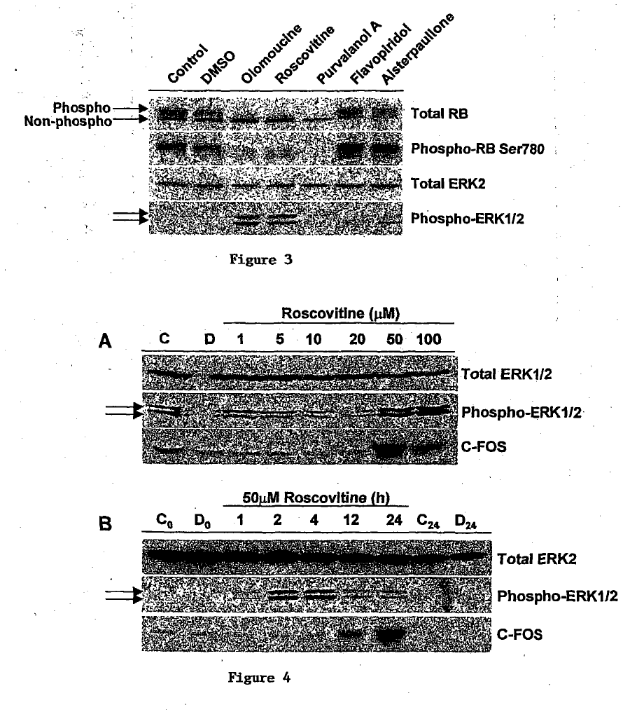

To investigate the possibility that the induction of ERK phosphorylation was a consequence of CDK inhibition, HT29 cells were treated with equitoxic (3×96 h IC₅₀) concentrations of R-roscovitine, olomoucine, purvalanol A, flavopiridol and alsterpaullone (FIG. 3). Olomoucine is a less potent relative of R-roscovitine with respect to inhibition of CDKs and cell growth. Purvalanol A is more potent as a CDK inhibitor and has a lower IC₅₀ for cell growth. The structurally dissimilar CDK inhibitors flavopiridol and alsterpaullone were included for comparison. The tri-substituted purines, R-roscovitine, olomoucine and purvalanol A all inhibited RB phosphorylation, consistent with CDK inhibition. Surprisingly, however, flavopiridol and alsterpaullone both failed to inhibit RB phosphorylation. As these compounds are known to have activity against the CDKs, this was an unexpected result, suggesting that additional loci of action may be responsible for the cell growth inhibition these compounds exhibit eg. Inhibition of GSK3β, which may actually promote RB phosphorylation through stabilisation of cyclin D1 (Diehl, J. A. et al. (1998). Genes and Development. 12:3499-3511). Both olomoucine and R-roscovitine induced ERK phosphorylation at 24 h whilst the more structurally dissimilar purvalanol A failed to induce ERK phosphorylation. Both flavopiridol and alsterpaullone had no effect upon ERK.

Induction of c-FOS Protein in Response to Activated ERK

HT29 cells were exposed to a range of concentrations of R-roscovitine for 24 h. At concentrations of 50 μM and greater, an increase in ERK phosphorylation was observed. At these same concentrations, an increase in c-FOS protein was detected by Western blotting, demonstrating functional activation of the ERK pathway (FIG. 4A). Exposure of HT29 s to a time course of 50 μM R-roscovitine resulted in an increase in phospho-ERK1/2 from 1 h, peaking at approximately 2-4 h and remaining above basal levels at 24 h. An increase of c-FOS protein-was detected from 12-24 h, subsequent to peak activation of the ERK pathway (FIG. 4B).

R-Roscovitine-induced ERK activation is functional and MEK-dependent

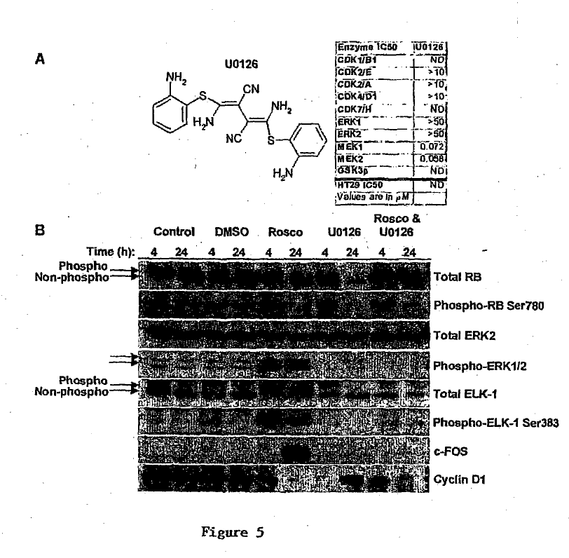

In order to determine the functional significance of ERK activation in response to R-roscovitine, HT29 cells were treated with R-roscovitine in combination with U0126 (FIG. 5A) (Favata, M. F. et al. (1998). Journal of Biological Chemistry. 273:18623-18632), a specific inhibitor of MEK1/2, and the phosphorylation of ELK-1 and expression of c-FOS was assessed by Western blotting (FIG. 5B). Phosphorylation of ELK-1 and increased expression of c-FOS are associated with functional activation of ERK. As observed previously, R-roscovitine caused a loss of RB phosphorylation at 24 h and an induction of ERK phosphorylation at 4-24 h. Interestingly, a strong induction of ELK-1 phosphorylation was observed after 4 h of R-roscovitine treatment, as shown by a mobility shift in total ELK-1 and confirmed by a phospho-specific antibody to Ser383. Further downstream, the activation of the ERK pathway is confirmed by the induction of c-FOS protein, which is transcriptionally regulated by ELK-1. These results demonstrated that the R-roscovitine-induced phosphorylation of ERK results in functional activation of the protein. It could be hypothesised that this may be an attempt by the cell to stimulate proliferation and override the anti-proliferative effect of R-roscovitine. Surprisingly, however, R-roscovitine inhibited the expression of cyclin D1 (FIG. 5B), transcription of which is thought to be regulated by the ERK pathway. It is possible that transcription of cyclin D1 may be increased but that cyclin D1 protein is proteolytically degraded. Alternatively, cyclin D1 transcription may be blocked through effects independent of the ERK pathway, potentially involving inhibition of CDKs.

To investigate the mechanism by which ERK phosphorylation was induced by R-roscovitine, simultaneous treatment with the MEK1/2 inhibitor U0126 was performed. At the routinely used concentration of 10 μM, U0126 prevented the induction of ERK phosphorylation in response to R-roscovitine and also blocked the phosphorylation of ELK-1 and the increase in c-FOS (FIG. 5B). Hence, it can be concluded that ERK phosphorylation and activation of the ERK pathway in response to R-roscovitine is MEK-dependent.

### Section 2

### RB Phosphorylation

Assessment of Methodology to Detect RB Phosphorylation by Western Blotting

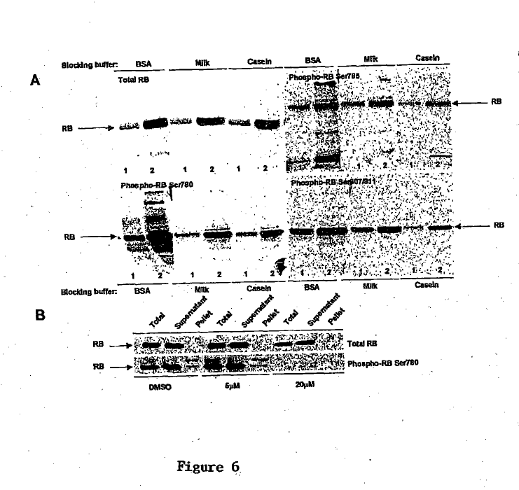

It is possible to detect phosphorylation of RB by the use of western blotting and antibodies to total RB, or to individual phosphorylation sites on RB that are consensus CDK sites. Experiments were undertaken using control, untreated cell lysates of HT29 and KM12 tumour cells to assess the sensitivity and specificity of commercially available antibodies against RB. Lysates were blotted after 6% SDS-PAGE and then blocked using either 3% BSA, milk or casein, dissolved in TBST. These blocking agents were also used as diluents for the antibodies during probing. The quality of blocking was assessed with respect to strength of signal when blots were developed and the amount of background signal present, lower being desirable. FIG. 6 shows that casein gives the clearest signal with low background but sufficient signal strength. The C-terminal (total) RB antibody and ser780 phospho-RB antibody demonstrated excellent consistency.

In order to determine how effective the lysis procedure was, HT29 cells were exposed to the CDK inhibitor purvalanol A for 24 h and then lysed in 100 μl lysis buffer per 1×10⁶ cells. The total lysate was split into two fractions, one labelled ‘total’. The remaining fraction was centrifuged at 14, 000 rpm to give the supernatant and pellet. The supernatant was transferred to a new tube. The pellet was resuspended in fresh lysis buffer. Equal volumes of lysate were analysed by western blotting and probed as shown in FIG. 6B. RB is present in the total and supernatant fractions but there is very little remaining in the pellet, hence demonstrating that the lysis procedure is efficient at extracting all the RB from the cells.

Roscovitine Causes a Growth Arrest in KM12 and HT29 Colon Tumour Cells

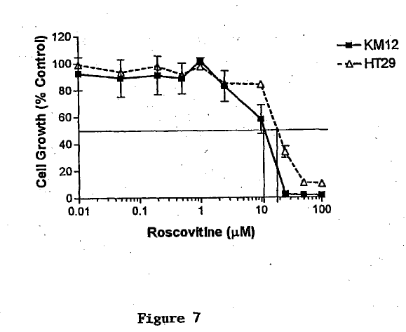

To determine the extent to which roscovitine inhibits cell growth, HT29 and KM12cells were seeded onto 96 well plates and treated with a range of doses of roscovitine. A plate was harvested every 24 h for 96 h and the total amount of protein in cells attached to the plate was determined by the SRB assay (FIG. 7). 96 h IC₅₀ values of 12 μM and 16 μM were determined for KM12 s and HT29 s respectively.

Roscovitine Causes a Dose and Time-Dependent Loss of RB Phosphorylation

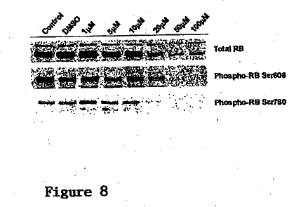

In order to assess the mechanism of growth inhibition of roscovitine, proliferating HT29 cells were treated with increasing doses of the compound for a period of 24 h. Samples were harvested for western blotting or for cell cycle analysis by FACS. A dose-dependent loss of RB phosphorylation was observed as shown by the thinning of the band for total RB. The C-terminal control antibody recognised all forms of RB, regardless of its phosphorylation status. The hypo-phosphorylated form of RB migrated more rapidly on SDS-PAGE than the phosphorylated forms, resulting in a low, thin band representing dephosphorylated RB. This was confirmed by using phospho-specific antibodies to single phosphorylation sites on RB. Since validating the original phospho-specific antibodies, a Ser608 antibody became available from Dr Sibylle Mittnacht. This site is potentially phosphorylated by CDK4/cyclin D1 and CDK2/cyclin A (Zarkowska and Mittnacht, J. Biol Chem (1997) 9:272 12738-46). A loss of phosphorylation was observed at sites Ser608 and Ser780 with 20 μM roscovitine and full inhibition was seen at 50 μM or greater (FIG. 8). When HT29cells were exposed to 50 μM roscovitine for different times, a loss of RB phosphorylation at Ser780 was observed after 24 h of exposure (FIG. 9). In order to ascertain whether the effects of roscovitine upon RB phosphorylation were conserved among different tumour cell lines, KM12 cells were exposed to different doses of roscovitine for 24 h (FIG. 10). As demonstrated in HT29 cells, a clear dephosphorylation of RB occurred with 20 μM roscovitine or greater after 24 h of exposure. A small reduction in phosphorylation at Ser780 was seen with as little as 10 μM, highlighting the greater sensitivity of this cell line.

Development of in vivo Assays for Measurement of RB Phosphorylation

Investigation into the action of roscovitine in animal models is required so that optimal dosing regimes and methods to monitor the efficacy of treatments can be established. Pharmacodynamic markers such as RB phosphorylation enable the anti-tumour effect and pharmacokinetic data to be correlated to modulation of the drug target. This has valuable potential in a clinical trial for optimising treatment schedules. It can also be valuable for directing analogue development. In order to determine if roscovitine could inhibit RB phosphorylation in vivo, nude mice were used as hosts for the KM12 xenograft. These mice were treated with roscovitine to evaluate anti-tumour effects and provide tumour material to assess RB phosphorylation. This can be used to determine if modulation of the target (ie. CDK2activity) occurs as it does in vitro. Also, lymphocytes were isolated from the mice and the level of RB phosphorylation assessed in order to provide a surrogate tissue that is easily accessible and may be a potential source of material in a human clinical trial. Removal of human tumour samples can be difficult and uncomfortable for patients, hence this is not ideal. If lymphocytes can be used as a surrogate tissue then inhibition of RB phosphorylation can be employed as a pharmacodynamic marker, thus providing information as to whether the compound is having an effect in the patients.

To determine if RB phosphorylation is detectable in mouse xenografts, one nude mouse with the LoVo xenograft was subjected to a 5Gy dose of radiation. A second control mouse, was untreated. Tumours were removed from the animals and homogenised in lysis buffer. The lysates were then subjected to SDS-PAGE and Western blotting to determine RB phosphorylation (FIG. 11). Following irradiation of the LoVo tumour, an inhibition of RB phosphorylation was detectable at Ser780, as expected. This was induced by DNA damage which initiates a G₁ arrest driven by TP53-induced transcription of p21^{WAF1/CIP1} (Brugarolas et al, Nature (1995) 377 552-7; Waldman et al, Cancer Res. (1995) 55 5187-90) which next in turn binds to and inhibits CDK2/cyclin E and CDK4/cyclin D. Therefore, it was possible to detect changes in RB phosphorylation by Western blotting of xenograft lysates.

To investigate whether RB phosphorylation was detectable in lymphocytes from mice and humans, lysates of a single mouse, 3 pooled mice and lymphocytes from a human volunteer were prepared. These were analysed by Western blotting and probed for total RB and phospho-RB at Ser780. FIG. 11 shows that whilst phospho-RB was detectable using the Ser780 antibody, total RB was not detectable in both mouse and human lymphocytes. A positive control of KM12 tumour cell lysates showed that this was not due to poor antibody hybridisation. Therefore, it, appeared that lymphocytes have low levels of RB expression and that the antibody Ser780 must have a greater affinity and thus be more sensitive than that to total RB.

It has been shown herein that the assay for RB phosphorylation was readily adaptable to studying material from in vivo sources.

1. A method of monitoring the activity of roscovitine comprising
(i) administering roscovitine to a cell, group of cells, an animal or human, and
(ii) detecting the presence of phosphorylated erk1/2.

2. A method according to claim 1, wherein roscovitine is administered to a mammal.

3. A method according to claim 1 or 2, wherein roscovitine is administered to a human.

4. A method according to claim 1, wherein the group of cells is a cell culture.

5. A method according to claim 5, wherein the cells are selected from HT29, KM12 and HCT116 cells.

6. A method according to claim 2, wherein the animal is an animal model a LoVo or KM12 xenograft mouse model.

7. A method according to any previous claim, wherein the presence of phosphorylated erk1/2 is detected in tumor cells or lymphocytes.

8. A method according to any preceding claim, wherein erk1/2 phosphorylation is monitored at least 24 hours after administration of roscovitine.

9. A method according to any preceding claim, wherein erk1/2 phosphorylation is monitored at least 48 hours after administration of roscovitine.

10. A method according to any preceding claim, wherein the level of phosphorylated erk1/2 is greater than that detected prior to administration of roscovitine.

11. A method according to any preceding claim, further comprising monitoring the level of phosphorylated retinoblastoma (RB) protein.

12. A method according to any preceding claim, wherein the level of phosphorylated retinoblastoma (RB) protein is less than that detected prior to administration of roscovitine.

13. A method according to any of claims 8 to 12 wherein the level of phosphorylated erk1/2 is monitored after 24 hours and the level of phosphorylated retinoblastoma (RB) protein is monitored at least 72 hours after administration of roscovitine.

14. A method of assessing suitable dose levels of roscovitine comprising monitoring the degree and rate of erk1/2 phosphorylation after administration of roscovitine to a cell, group of cells, animal model or human.

15. A method according to claim 14, further comprising correlating the degree and rate of erk1/2 phosphorylation with the known rate of inhibition of either CDK2 or RB phosphorylation by roscovitine at the same dosage, over the same time period.

16. A method of monitoring the activity of roscovitine in a cassette dosing assay whereby a cocktail of roscovitine and other CDKI's are administered together and roscovitine activity is monitored in accordance with a method of claim 1 to 13.

17. A method of identifying a candidate drug having roscovitine-like activity comprising administering said candidate drug to cell, group of cells, animal model or human and monitoring the presence or absence of erk1/2 phosphorylation.

18. A method according to any preceding claims, wherein roscovitine is R-roscovitine.

19. Use of phospho-erk 1 and/or phospho-erk 2 in the monitoring of activity of roscovitine.

20. Use according to claim 19, wherein the presence of phosphorylated erk 1 and/or erk 2 is monitored after the administration of roscovitine to a cell, group of cells, an animal model or human.

21. Use according to claim 19 or 20, wherein roscovitine is R-roscovitine.

22. A kit for assessing the activity of roscovitine comprising antibodies for at least one of phospho-erk1 or phospho-erk2 and optionally antibodies for RB (whole), RB Ser780 or RB Ser608.

23. A kit according to claim 22, wherein the antibodies are for phospho-erk1 or phospho-erk2 alone or in combination with one of the RB antibodies.

24. A kit according to claim 23, wherein the RB antibody is for RB Ser608.

25. A kit according to any of claims 22-24, wherein roscovitine is R-roscovitine.

26. Use of a kit as defined in any of claims 20 to 22, in a method defined in any of claims 1 to 13.
